(19) United States
(12) Patent Application Publication
(10) Pub. No.: US 2021/0102839 A1
(43) Pub. Date: Apr. 8, 2021

(54) SUNSCREEN DETECTION APPARATUS AND METHOD

(71) Applicant: Beijing Youcai Technology Co., Ltd., Beijing (CN)

(72) Inventor: Xiaoyong PAN, Beijing (CN)

(73) Assignee: Beijing Youcai Technology Co., Ltd., Beijing (CN)

(21) Appl. No.: 17/105,641

(22) Filed: Nov. 27, 2020

Related U.S. Application Data
(63) Continuation-in-part of application No. PCT/CN2018/094975, filed on Jul. 9, 2018.

(30) Foreign Application Priority Data
May 29, 2018 (CN) 201810531040.7
May 29, 2018 (CN) 201820817674.4

(51) Int. Cl. G01J 1/42; G06T 7/00; G16H 70/40

(52) U.S. Cl. CPC G01J 1/429; G06T 7/0012; A61Q 17/04; G06T 2207/30088; G16H 70/40

(57) ABSTRACT

The disclosure relates to an apparatus including a UV camera configured for capturing reflected ultraviolet light of the subject and outputting image data, wherein the surface of the subject includes at least one part of an area covered by a sunscreen substance or not covered by the sunscreen substance; a UV detector coupled to the UV camera, for detecting the ambient ultraviolet intensity around the subject; and a supplement light configured to be activated according to the ultraviolet intensity detected by the UV detector.

### CROSS-REFERENCE TO RELATED APPLICATION

This application is a continuation-in-part application of international PCT Application No. PCT/CN2018/094975, filed on Jul. 9, 2018, which claims the priority benefit of Chinese Patent Application No. 201810531040.7, and Chinese Patent Application No. 201820817674.4, filed on May 29, 2018. The entirety of each of the above mentioned patent applications is hereby incorporated by reference herein and made a part of this specification.

### TECHNICAL FIELD

The technical field of the disclosure relates to sunscreen detection, and more particularly, to a sunscreen detection apparatus and method.

### BACKGROUND

More than 10 million people are diagnosed with skin cancers worldwide every year. The majority of the cases are directly associated with exposure to ultraviolet (UV) radiation from the sun. The most effective way to prevent ultraviolet damage is by applying sunscreen. The protection and duration provided by the sunscreen vary from skin types and products. Moreover, the user cannot easily visualize the effective coverage of sunscreen on the skin and also cannot recognize whether the sunscreen being worn out.

In addition, the user cannot directly verify the anti-ultraviolet performance of automobile films and coated window glasses, that the performance may degrade over time.

### SUMMARY

An aspect of the disclosure provides an apparatus comprising: a UV camera configured for capturing reflected ultraviolet light of the subject and for outputting image data, wherein the surface of the subject includes at least one part of an area covered or an area not covered by the sunscreen substance; a UV detector coupled to the UV camera, for detecting the ambient ultraviolet intensity around the subject; and a supplement light configured to be activated according to the ultraviolet intensity detected by the UV detector.

Another aspect of the disclosure provides an apparatus comprising: a container for containing sunscreen substance; a nozzle coupled to the container for spraying the sunscreen substance; a UV camera coupled to a controller for capturing reflected ultraviolet light of the subject and outputting image data, wherein the surface of the subject includes at least one part of an area covered or an area not covered by the sunscreen substance; a UV detector coupled to the UV camera, for detecting the ambient ultraviolet intensity around the subject; a controller coupled to the nozzle, for receiving and responding to the trigger command and for triggering the nozzle to start spraying sunscreen substance onto the area not covered by the sunscreen substance.

A further aspect of the disclosure provides a method comprising: receiving, by a mobile device, ambient ultraviolet intensity around the subject by a UV detector, wherein the surface of the subject includes at least one part of an area covered or not covered by the sunscreen substance; displaying, on the mobile device, an image of the subject based on the output of the UV camera, in such manner of reflecting the effective sunscreen coverage; and obtaining, by the mobile device, corresponding calibrated sunscreen effective duration on the subject according to multiple iterations of detected sunscreen effective duration from each image captured at a given SPF or PA value of the sunscreen.

### BRIEF DESCRIPTION OF THE DRAWINGS

FIG. 1 is a block diagram of an apparatus according to an embodiment of the disclosure.

FIG. 2 is a block diagram of an apparatus according to an embodiment of the disclosure.

FIG. 3 is a flow diagram of a method according to an embodiment of the disclosure.

### DETAILED DESCRIPTION OF THE EMBODIMENTS

The embodiments listed below are written only to illustrate the applications of this apparatus and method and not to limit the scope. The equivalent form of modifications towards this apparatus and method shall be categorized as within the scope the claims.

In the following discussion and claims, the terms “comprising” and “including” are interpreted as “including but not limited to . . . ”. In addition, the term “coupled” is intended to mean an indirect or direct connection.

Please note that the positions of the modules in the drawings are only illustrative, and the disclosure does not intend to limit the scope of the claims to the specific positions of the modules in the drawings.

The terms “mobile device” and “controller” referred to in the disclosure may be used interchangeably. In addition, “container” and “sunscreen bottle” can be used interchangeably, and “sunscreen substance” and “sunscreen” can be used interchangeably.

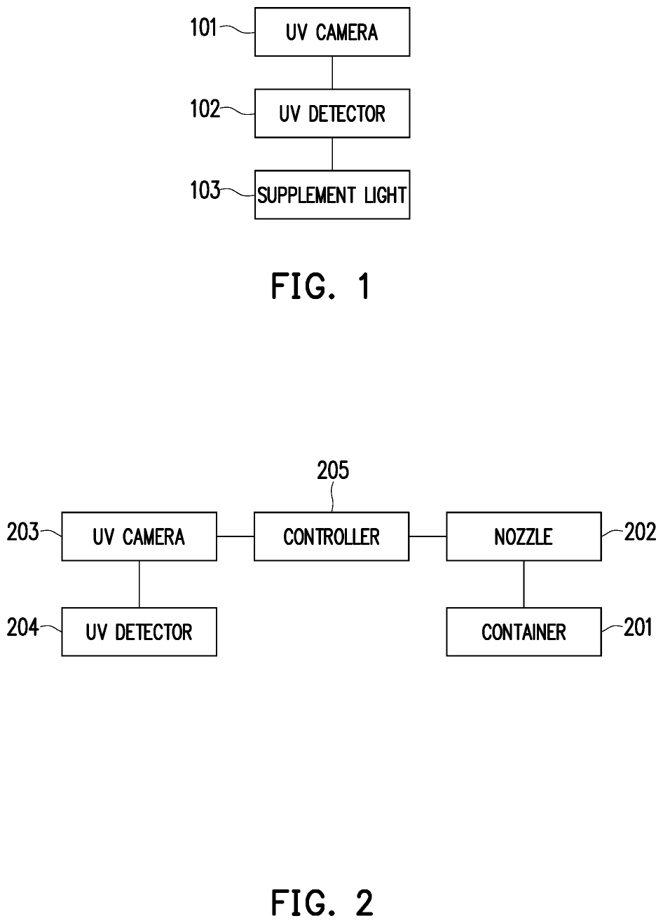

FIG. 1 shows an apparatus according to an embodiment, including: a UV camera 101 configured for capturing reflected ultraviolet light of the subject and outputting image data, wherein the surface of the subject includes at least one part of an area covered or an area not covered by the sunscreen substance; a UV detector 102 coupled to the UV camera, for detecting the ambient ultraviolet intensity around the subject; and a supplement light 103 configured to be activated according to the ultraviolet intensity detected by the UV detector.

FIG. 2 shows an apparatus according to an embodiment including: a container 201 for containing sunscreen substance; a nozzle 202 coupled to the container for spraying the sunscreen substance; a UV camera 203 coupled to a controller for capturing reflected ultraviolet light of the subject and outputting image data, wherein the surface of the subject includes at least one part of an area covered or not covered by the sunscreen substance; a UV detector 204 coupled to the UV camera, for detecting the ambient ultraviolet intensity around the subject; a controller 205 coupled to the nozzle, for receiving and responding to the trigger command and for triggering the nozzle to start spraying sunscreen substance onto the area not covered by the sunscreen substance.

The following is a detailed description of how each component works.

### UV Detector and Supplement Light

The UV detector is detachably mounted to the UV camera and is adapted to be communicatively connected to the mobile device. The UV detector is used for detecting the ambient ultraviolet intensity around the subject. The sunscreen detection apparatus includes a supplement light. The illumination direction of the supplement light is substantially the same as the orientation of the UV camera.

Since the operation of the UV camera is based on the principle of ultraviolet imaging, its operation requires sufficient ultraviolet of the environment. When the ambient ultraviolet intensity is too low, the accuracy of the detection by the UV camera is affected.

Based on this, the mobile device can also activate the UV detector to detect the ultraviolet around the subject and acquire the real-time ambient ultraviolet intensity, and can then activate the supplement light simultaneously. In one embodiment, in response to receiving a user-initiated request to detect ultraviolet intensity, the application software may issue a request to activate the UV detector and the supplement light.

By displaying the acquired real-time ultraviolet intensity on the screen of the mobile device, the user can know the ultraviolet intensity of the environment in which the current subject is located. Thus, the user can determine whether to activate the UV camera. If the ambient ultraviolet intensity is low, the user can activate the supplement light to emit ultraviolet radiation to the subject. In addition, the apparatus can also automatically activate the UV camera and or the supplement light. For example, the detected real-time ultraviolet intensity is compared with a threshold value pre-stored in the mobile device. When the real-time ultraviolet light intensity is greater than a threshold value, the UV camera is activated without the supplement light. When the real-time ultraviolet intensity is less than the threshold value, the UV camera is activated with the supplement light.

### UV Camera

In one embodiment, the UV camera may include a camera, but the disclosure is not limited and may include any apparatus capable of identifying a subject. In one embodiment, the UV camera may include at least one of a UV detector and a supplement light.

A UV camera is detachably mounted to the mobile device and adapted to communicate with the mobile device. The UV camera may be connected to the mobile device through an interface. The interface includes a USB or lightning interface, for example, a Type-C USB connector. Through this interface, the UV camera can transmit the acquired image or data to the controller of the mobile device, which can transmit control commands to the UV camera.

Since the operation of the UV camera is based on the operation principle of ultraviolet imaging, it requires sufficient ultraviolet of the environment. When the ambient ultraviolet intensity is too low, the accuracy of the detection by the UV camera is affected. Therefore, it is necessary to activate the UV detector before activating the UV camera and to activate the supplement light when the ambient ultraviolet intensity is low.

### Container

The apparatus also includes a container. The container may be a sunscreen bottle detachably mounted to a UV camera or a UV detector. In one embodiment, the container is a roller bottle or spray bottle. The use of a roller bottle and a spray bottle makes the applying or spraying of sunscreen more uniformly.

### Subject

The captures of a UV camera are displayed on the screen of the mobile device in the form of an image or video by detecting the absorption and reflection ability of ultraviolet radiations of the subject, which can be the skin, paint, coated glass, filmed glass, tinted glass, etc.

In one embodiment, the repainted area on the automobile body can be distinguished by using this disclosure apparatus or method. Since the ultraviolet absorption and reflection ability of the repainted area is different from the paint done at other times, the repainted area can be easily discovered by the different appearance under the UV camera.

### Display Pattern

The user can view patterns of the current sunscreen coverages on the mobile device. For example, where chemical sunscreen absorbs ultraviolet, there are not ultraviolet reflections on the covered area, and therefore, the image of such area is displayed as dark or black. On the contrary, physical sunscreen reflects ultraviolet, the image of such area covered by physical sunscreen is displayed as light or white.

In addition, it is possible to display the traces and coverages of sunscreen protection on the subject on the mobile device. In one embodiment, a real-time or recorded grayscale image or video may be displayed on the mobile device. The real-time grayscale image reflects the degree of current sunscreen efficiency and coverage on the subject. The user then may apply sunscreen to the under-protected area accordingly.

### Trigger Command

This embodiment can also determine whether the sunscreen protection performance on the skin is sufficient and can also identify which skin area is required to apply sunscreen. The user can improve the designated area and review with the UV camera again.

In one embodiment, a nozzle is configured to receive a trigger command from the controller to spray a sunscreen substance onto the area not sufficiently covered by the sunscreen substance.

### Controller

The controller calculates the effective duration of the sunscreen coverage and transmits the trigger command to the nozzle. The effective duration is calibrated with multiple iterations of detected sunscreen effective duration from each image captured at a given sunscreen.

In this disclosure, the controller may be allocated inside the nozzle but is not limited. The controller may also be allocated outside of the nozzle, as long as the operation of the nozzle control can be achieved.

In one embodiment, the nozzle is activated to spray the sunscreen substance onto an area which is not effectively covered by the sunscreen substance. In order to make the spraying more directive, a UV camera can be used to provide more directing information. In one embodiment, more directing information may be provided on the application software interface. For example, a user may manually specify a designated area according to a grayscale image.

In one embodiment, the nozzle may further include a steering unit coupled to the controller. Using the directing information, the steering unit is controlled to rotate and to direct the nozzle towards the designated area where the sunscreen substance should be sprayed. In one embodiment, the steering unit may be controlled with a user interface of the application software.

### Calculation of Sunscreen Effective Duration

In one embodiment, an SPF value or PA value of sunscreen is inputted from the user.

The initial time when sunscreen is first applied onto the subject with the UV camera capturing is set as T0. The interval time ΔT from the initial time. The subject is then captured with a UV camera at different times. For example, at times T1 (T0+ΔT1), T2 (T0+ΔT2), T3 (T0+ΔT3) . . . Tn (T0+ΔTn), the image of the subject is captured and analyzed. The time Tn that when an area of the subject is firstly analyzed or manually inputted as insufficient protection is the sunscreen effective duration, Tn-T0 or ΔTn, with reference to a given SPF or PA value.

The sunscreen effective duration is recorded in the application software database. Multiple usages of sunscreen may result in multiple iterations of the sunscreen effective duration with reference to given SPF or PA values respectively. In one embodiment, an estimated sunscreen effective duration may also be provided for a user based on an SPF or a PA value. When the protected subject is checked with the UV camera for effectiveness, the effective duration is compared and confirmed. If the image and the estimated duration is not confirmed, the new sunscreen effective duration ΔTn is calibrated or updated.

In one embodiment, multiple most recent measurements of the sunscreen effective durations may be averaged. The application software can remind the user to reapply the sunscreen through an alarm sound or a screen display of the mobile device before a calibrated time (e.g., 10 minutes) before sunscreen is worn out.

This solution takes into account different conditions for different subjects or users (different rates of absorption of the sunscreen by different users' skin) and the influence of the user's activities. For example, the effective duration may be strongly reduced for outdoor activities comparing to indoor activities.

In one embodiment, the sunscreen effective duration may be adjusted according to the skin types, the weather, or manual control.

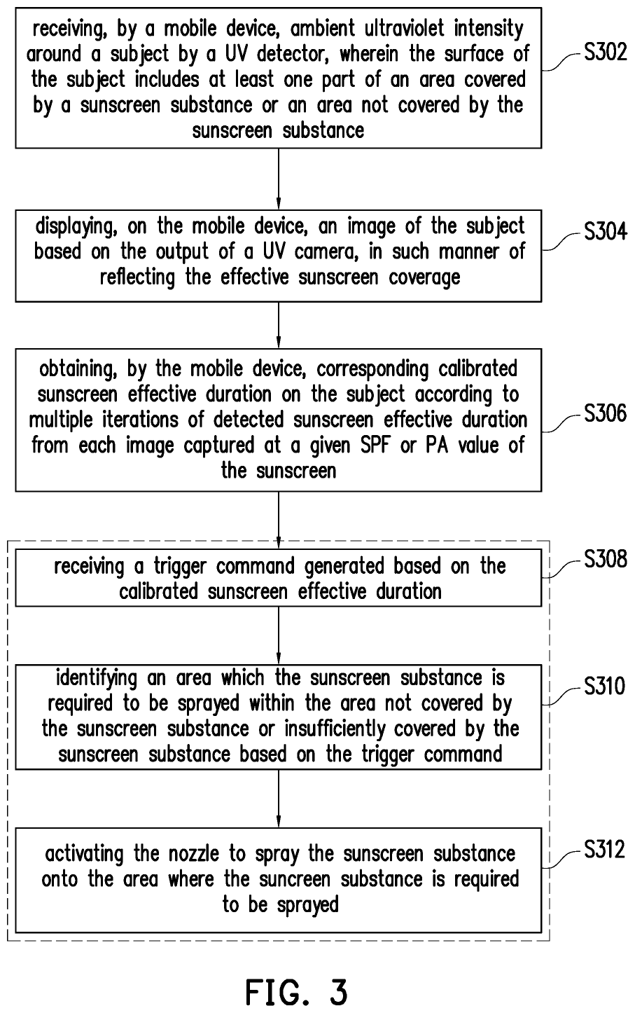

FIG. 3 shows a method according to an embodiment. The method includes steps S302 to S306 as follows:

step S302, receiving, by a mobile device, ambient ultraviolet intensity around the subject by a UV detector, wherein the surface of the subject includes at least one part of an area covered by a sunscreen substance or an area not covered by the sunscreen substance;

step S304, displaying, on the mobile device, an image of the subject based on the output of the UV camera, in such manner of reflecting the effective sunscreen coverage; and

step S306, obtaining, by the mobile device, corresponding calibrated sunscreen effective duration on the subject according to multiple iterations of detected sunscreen effective duration from each image captured at a given SPF or PA value of the sunscreen.

In an embodiment, the method further includes steps S308 to S312.

At step S308, a trigger command may be received based on the calibrated sunscreen effective duration.

At step S310, an area which the sunscreen substance should be applied may be designated. That is, step S310 may include identifying an area which the sunscreen substance is required to be sprayed within the area not covered by the sunscreen substance or insufficiently covered by the sunscreen substance based on the trigger command.

At step S312, the nozzle may be activated to spray the sunscreen substance onto the designated area (the area which the sunscreen substance is required to be sprayed).

In one embodiment, the designated area in which the sunscreen substance should be applied or sprayed refers to an area on the subject that is covered by non or insufficient sunscreen substance.

In one embodiment, the above described method may be implemented in part or in whole in the controller. The method may also be implemented in part or in whole in the application software of the mobile device. The functions may be implemented in both of the controller and application software of the mobile device.

The various parts have been described in detail in the above context, and will not be repeated in the followings to avoid obscuring the disclosure.

Persons skilled in the art will understand the illustrative block charts, modules, elements, components, methods, and algorithms described herein can be implemented as electronic hardware, software, or a combination of both. To illustrate this interchangeability of hardware and software, illustrative block charts, modules, elements, components, methods, and algorithms have been generally described above according to their functions. Whether this function is implemented as hardware or software depends on the specific application and design constraints imposed on the entire system. Those persons skilled in the art can implement the described functions in different ways for a different application. Without departing from the scope of the present technology, various components and blocks may be arranged differently (for example, arranged in a different order, or divided differently).

It is understood that the specific order or hierarchy of steps in the processes disclosed is an illustration of an example method. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The foregoing description is provided to enable any person skilled in the art to practice the various aspects described herein. The foregoing description provides various examples of the present technology, and the present technology is not limited to these examples. Various modifications to these aspects will be obvious to those skilled in the art, and the general principles defined here can be applied to other aspects. Therefore, the claims are not intended to be limited to the aspects shown herein, but should be given a full scope consistent with the language of the claims, wherein unless specifically stated otherwise, elements mentioned in the singular are not intended to mean “one and only one”, rather “one or more”. Unless specifically stated otherwise, the term “some” refers to one or more. The title and subtitle, if any, are for convenience only and do not limit the invention.

A phrase such as an “aspect” does not imply that such aspect is essential to the subject technology or that such aspect applies to all configurations of the subject technology. A disclosure relating to an aspect may apply to all configurations or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations or one or more configurations. A configuration may provide one or more examples. A phrase such as a “configuration” may refer to one or more configurations and vice versa.

The word “example” is used herein to mean “serving as an example or illustration.” Any aspect or design described herein as “example” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”. Furthermore, to the extent that the term “includes” and “have”, or the like is used in the description or the claims, such term is intended to be inclusive in a manner similar to the term “include” as “include” is interpreted when employed as a transitional word in a claim.

References to “one embodiment”, “an embodiment”, “some embodiments”, “various embodiments” or the like indicate that a particular element or characteristic is included in at least one embodiment of the invention. Although the phrases may appear in various places, the phrases do not necessarily refer to the same embodiment. In conjunction with the disclosure, those skilled in the art will be able to design and incorporate any one of the varieties of mechanisms suitable for accomplishing the above described functionalities.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. An apparatus, comprising:
a UV camera configured for capturing reflected ultraviolet light of a subject and outputting image data, wherein a surface of the subject includes at least one part of an area covered by a sunscreen substance or an area not covered by the sunscreen substance;
a UV detector coupled to the UV camera, for detecting an ambient ultraviolet intensity around the subject; and
a supplement light configured to be activated according to the ambient ultraviolet intensity detected by the UV detector.

2. The apparatus of claim 1, wherein the sunscreen substance includes a sunscreen.

3. The apparatus of claim 1, wherein the sunscreen substance includes a skin, a paint, a coated glass, a filmed glass, or a tinted glass.

4. An apparatus, comprising:
a container for containing a sunscreen substance;
a nozzle coupled to the container for spraying the sunscreen substance;
a UV camera coupled to a controller for capturing reflected ultraviolet light of a subject and outputting image data, wherein the surface of the subject includes at least one part of an area covered by the sunscreen substance or an area not covered by the sunscreen substance;
a UV detector coupled to the UV camera, for detecting an ambient ultraviolet intensity around the subject; and
the controller coupled to the nozzle, for receiving and responding to a trigger command and for triggering the nozzle to start spraying sunscreen substance onto the area not covered by the sunscreen substance.

5. The apparatus of claim 4, wherein the sunscreen substance includes a sunscreen.

6. The apparatus of claim 4, wherein the sunscreen substance includes a skin, a paint, a coated glass, a filmed glass, or a tinted glass.

7. A method, comprising:
receiving, by a mobile device, an ambient ultraviolet intensity around a subject by a UV detector, wherein a surface of the subject includes at least one part of an area covered by a sunscreen substance or an area not covered by the sunscreen substance;
displaying, on the mobile device, an image of the subject based on an output of a UV camera, in such manner of reflecting an effective sunscreen coverage; and
obtaining, by the mobile device, corresponding a calibrated sunscreen effective duration on the subject according to multiple iterations of detected sunscreen effective duration from each image captured at a given SPF or PA value of the sunscreen.

8. The method of claim 7, further comprising:
receiving a trigger command generated based on the calibrated sunscreen effective duration;
identifying an area which the sunscreen substance is required to be sprayed within the area not covered by the sunscreen or an area insufficiently covered by the sunscreen substance based on the trigger command; and
activating the nozzle to spray the sunscreen substance onto the area which the sunscreen substance is required to be sprayed.

9. The method of claim 7, wherein the displaying includes displaying in a manner of at least one of a grayscale image, a grayscale video, or a grayscale real-time video.

10. The method of claim 7, further comprising:
displaying the area not covered by the sunscreen substance as light gray or white and displaying the area covered by the sunscreen substance as dark gray or black.

11. The method of claim 7, wherein the sunscreen substance includes a sunscreen.

12. The method of claim 7, wherein the sunscreen substance includes a skin, a paint, a coated glass, a filmed glass, or a tinted glass.
